(19) United States
(12) Patent Application Publication
(10) Pub. No.: US 2025/0011857 A1
(43) Pub. Date: Jan. 9, 2025

(54) Detecting Varying Conductance Indicative of Sequential Interactions Between Nucleobases

(71) Applicant: Digital Biotechnologies, Inc., Seattle, WA (US)

(72) Inventors: Jason H. Bielas, Seattle, WA (US); Harlan S. Robins, Seattle, WA (US)

(21) Appl. No.: 18/708,182

(22) PCT Filed: Nov. 9, 2022
(86) PCT No.: PCT/US2022/049451

§ 371 Date: May 7, 2024

Related U.S. Application Data
(60) Provisional application No. 63/263,803, filed on Nov. 9, 2021.

(51) Int. Cl. C12Q 1/6869; G01N 27/447

(52) U.S. Cl. CPC C12Q 1/6869; G01N 27/4473; G01N 27/44791

(57) ABSTRACT

Provided are methods comprising causing relative movement of a first nucleic acid through an opening formed at least in part by an electrolyzed second nucleic acid. Such methods comprise, during the relative movement, detecting a varying conductance along the first nucleic acid, or a varying conductance between the first nucleic acid and an electrode proximate to the first nucleic acid, wherein the varying conductance is indicative of sequential interactions between nucleobases of the first nucleic acid and one or more nucleobases of the electrolyzed second nucleic acid. The varying conductance comprises conductance fingerprints for the different nucleobases in the first nucleic acid. In certain embodiments, the methods comprise determining the identity of one or more nucleotide, optionally determining a sequence, of the first nucleic acid based on the varying conductance. Computer-readable media and systems that find use, e.g., in practicing the methods of the present disclosure, are also provided.

### CROSS-REFERENCE TO RELATED APPLICATIONS

This application claims the benefit of priority of U.S. Provisional Patent Application No. 63/263,803, filed Nov. 9, 2021, the disclosure of which application is incorporated herein by reference in its entirety.

### INTRODUCTION

Nucleic acid sequencing is the process of determining the order of nucleotides in a nucleic acid sequence (e.g., DNA, RNA), and has become a fundamental process in fields such as medical diagnostics, forensic biology, and biological research. Available techniques for sequencing have included, e.g., the Sanger method. This procedure involves random incorporation of chain-terminating dideoxynucleotides by DNA polymerase during in vitro DNA replication. In addition, next-generation sequencing platforms (sometimes referred to as “second generation” sequencing) use different technologies for sequencing, such as pyrosequencing, sequencing by synthesis, sequencing by ligation, or nanopore-based sequencing. However, new sequencing methodologies are desirable to address issues related to cost, sequencing quality, sequencing time, efficiency, and ease of use.

Recently, sequencing methods exploiting the electrochemical properties of nucleic acids such as DNA have been proposed. In particular, approaches have been contemplated in which DNA is electrically driven through nanopores. As the DNA translocates through the nanopore, it may be detected by measuring an ionic blockade current or transverse current. However, nanopore sequencing methods using electrochemical properties of nucleic acids have yet to reach widespread adoption due to issues related to feasibility, accuracy and cost. Alternative electrochemical sequencing approaches are consequently desirable.

### SUMMARY

Provided are methods comprising causing relative movement of a first nucleic acid through an opening formed at least in part by an electrolyzed second nucleic acid. Such methods comprise, during the relative movement, detecting a varying conductance along the first nucleic acid, or a varying conductance between the first nucleic acid and an electrode proximate to the first nucleic acid, wherein the varying conductance is indicative of sequential interactions between nucleobases of the first nucleic acid and one or more nucleobases of the electrolyzed second nucleic acid. In certain embodiments, the varying conductance comprises conductance fingerprints for the different nucleobases in the first nucleic acid. According to some embodiments, the methods comprise determining the identity of one or more nucleotides of the first nucleic acid based on the varying conductance. In certain embodiments, the methods comprise determining a nucleotide sequence of the first nucleic acid based on the varying conductance. Computer-readable media and systems that find use, e.g., in practicing the methods of the present disclosure, are also provided.

The methods, system and computer-readable media of the present disclosure are based in part on the inventors' recognition that different nucleotides having different nucleobases—e.g., adenine (A), thymine (T), guanine (G), cytosine (C), uracil (U), or non-natural variants thereof—and their various sequences, each with a distinct chemical composition and structure, can be associated with a specific signature of transverse tunneling (perpendicular to the DNA or RNA axis). In support of this, it has been demonstrated that DNA base pairs behave as biological Aviram-Ratner electrical rectifiers because of the spatial separation and weak bonding between the nucleobases, and because the current that flows across these base pairs varies based on the specific nature of the particular nucleotide (see, e.g., Agapito et al. Nanotechnology, 23(13), 135202). In other words, the DNA base pair can serve as a one-way conductor of electric current, and interactions between given nucleobases are characterized by a particular “conductance fingerprint”. Such is shown in FIG. 1A-1D (adapted from Agapito et al. (supra)). Top and side views of the C-G (FIG. 1A) and T-A (FIG. 1C) junctions are shown, along with density of states projected on the purine and pyrimidine components of the C-G (FIG. 1B) and T-A (FIG. 1D) junctions.

The present inventors have determined that DNA sequencing can be carried out by evaluating conductance for the presence of conductance fingerprints as a nucleic acid to be sequenced is moved relative to a second nucleic acid.

### BRIEF DESCRIPTION OF THE FIGURES

FIG. 1A-1D depict a “conductance fingerprint” associated with certain interactions between given nucleobases.

FIG. 2 presents a schematic illustration of a method according to embodiments of the present disclosure comprising relative movement of a first nucleic acid through a plurality of openings formed by a plurality of electrolyzed nucleic acids.

FIG. 3 presents a schematic illustration of a method according to embodiments of the present disclosure comprising relative movement of a first nucleic acid through a plurality of openings formed by a plurality of electrolyzed nucleic acids.

FIG. 4 presents a schematic illustration of a method according to embodiments of the present disclosure comprising relative movement of a first nucleic acid through a plurality of openings formed by a plurality of electrolyzed nucleic acids.

FIG. 5 presents a schematic illustration of various electrolyzed nucleic acid configurations that find use in practicing the methods of the present disclosure.

FIG. 6 presents a schematic illustration of embodiments in which a varying conductance between the first nucleic acid and an electrode proximate to the first nucleic acid is detected, and the varying conductance is indicative of sequential interactions between nucleobases of the first nucleic acid and one or more nucleobases of electrolyzed nucleic acids in the form of bridge structures.

FIG. 7A-7B present schematic illustrations of embodiments in which a varying conductance between the first nucleic acid and an electrode proximate to the first nucleic acid is detected, and the varying conductance is indicative of sequential interactions between nucleobases of the first nucleic acid and one or more nucleobases of electrolyzed nucleic acids.

FIG. 8 depicts an embodiment of the invention in which a varying conductance can be detected between a first nucleic acid and an electrode proximate to the first nucleic acid. “A”=ammeter.

FIG. 9A-9B depict the architecture of a chip for use in the subject methods according to certain embodiments.

FIG. 10 presents a micrograph showing an insulation layer between right and left electrodes and a gate electrode, according to certain embodiments.

FIG. 11A-11C present micrographs taken via helium ion microscopy of a chip for use in the subject methods.

FIG. 12 depicts a prototype of a chip constructed as shown in FIG. 9A-9B.

FIG. 13A-13D present micrographs of the chip electrodes that were obtained via atomic force microscopy (AFM).

FIG. 14A-14E depict topographic and conductive aspects of the chip prototype constructed as shown in FIG. 9A-9B.

FIG. 15A-15B present AFM images showing single stranded DNA immobilized on a gold substrate via thio bonds.

### DETAILED DESCRIPTION

Before the methods, systems and computer-readable media of the present disclosure are described in greater detail, it is to be understood that the methods, systems and computer-readable media are not limited to particular embodiments described, as such may, of course, vary. It is also to be understood that the terminology used herein is for the purpose of describing particular embodiments only, and is not intended to be limiting, since the scope of the methods, systems and computer-readable media will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, systems and computer-readable media. The upper and lower limits of these smaller ranges may independently be included in the smaller ranges and are also encompassed within the methods, systems and computer-readable media, subject to any specifically excluded limit in the stated range. Where the stated range includes one or both of the limits, ranges excluding either or both of those included limits are also included in the methods, systems and computer-readable media.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, systems and computer-readable media belong. Although any methods, systems and computer-readable media similar or equivalent to those described herein can also be used in the practice or testing of the methods, systems and computer-readable media, representative illustrative methods, systems and computer-readable media are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, systems and computer-readable media are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, systems and computer-readable media, which are, for clarity, described in the context of separate embodiments, may also be provided in combination in a single embodiment. Conversely, various features of the methods, systems and computer-readable media, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, systems and computer-readable media and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Definitions

The term “nucleotide” is intended to include those moieties that contain not only the naturally occurring purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

As used herein, an “oligonucleotide” is a single-stranded multimer of nucleotides from 2 to 500 nucleotides, e.g., 2 to 200 nucleotides. Oligonucleotides may be synthetic or may be made enzymatically, and, in some embodiments, are 5 to 50 nucleotides in length (e.g., 9 to 50 nucleotides in length). Oligonucleotides may contain ribonucleotide monomers (i.e., may be oligoribonucleotides or “RNA oligonucleotides”) or deoxyribonucleotide monomers (i.e., may be oligodeoxyribonucleotides or “DNA oligonucleotides”). Oligonucleotides may be 5 to 9, 10 to 20, 21 to 30, 31 to 40, 41 to 50, 51 to 60, 61 to 70, 71 to 80, 80 to 100, 100 to 150 or 150 to 200, up to 500 or more nucleotides in length, for example.

The terms “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, greater than 10,000 bases, greater than 100,000 bases, greater than about 1,000,000, up to about 10¹⁰ or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylenecarbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid,” or “UNA,” is a nucleic acid containing non-natural nucleotides that bind to each other with reduced stability. For example, an unstructured nucleic acid may contain a G residue and a C residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is incorporated by reference herein for disclosure of UNA.

### Methods

As summarized above, aspects of the present disclosure include methods comprising detecting a varying conductance along a first nucleic acid, or between a first nucleic acid and an electrode proximate to the first nucleic acid, which varying conductance may be measured and utilized to sequence the first nucleic acid. As will be appreciated upon review of the present disclosure, the present methods constitute an improvement over existing sequencing technologies, e.g., because the present methods may be performed much more rapidly at decreased cost (e.g., essentially no consumables) with increased accuracy and portability compared to existing approaches for nucleic acid sequencing.

According to some embodiments, the methods comprise causing relative movement of a first nucleic acid (e.g., a nucleic acid to be sequenced) through an opening formed at least in part by an electrolyzed second nucleic acid. By “causing relative movement”, it is meant that at least one of the first nucleic and electrolyzed second nucleic acid changes location relative to the other. In some versions of the subject methods, the first nucleic acid moves while the second nucleic acid remains stationary. In other embodiments, the second nucleic acid moves while the first nucleic acid remains stationary. In still other embodiments, both of the first and second nucleic acids move. Where methods include causing the movement of the first nucleic acid relative to the second nucleic acid, the movement may be effected using any convenient approach. For example, methods may include pulling the first nucleic acid through the opening formed at least in part by the second nucleic acid. By “pulling”, it is meant exerting a force on the nucleic acid sufficient to cause that nucleic acid to change location. The requisite force may exist in any convenient form. For example, in some cases, the first nucleic acid is attached to an elongate structure which can be used to pull (i.e., physically pull) said nucleic acid. The force may be driven using any convenient approach, including but not limited to chemical propulsion, magnetic propulsion, ultrasound-driven propulsion, light-driven propulsion, electrically-driven propulsion, and combinations thereof. Nanoscale methods of propulsion are described in, e.g., Wang et al. Chemical reviews, 115(16), 8704-8735; herein incorporated by reference in its entirety. The speed of the relative movement may vary. In some instances, the speed of the relative movement ranges from 1 to 1000 nucleotides/second.

In certain versions, the elongate structure is a nanowire. Nanowires may have any convenient diameter, such as where the diameter ranges from 0.5 nm to 500 nm, such as 1 nm to 200 nm, and including 5 nm to 100 nm. The nanowire may comprise any convenient material. Exemplary nanowire materials include, but are not limited to, carbon, germanium, silicon, gold, copper, yttrium barium copper oxide (YBCO), indium phosphide, gallium nitride, nickel, platinum, combinations thereof, and the like.

In additional embodiments, the elongate structure is a nanotube. A nanotube is a tube-like structure generally comprised of carbon (e.g., fullerene, graphene). Various techniques for producing carbon nanotubes have been developed. As examples, methods of forming carbon nanotubes are described in U.S. Pat. Nos. 5,753,088 and 5,482,601, the disclosures of which are hereby incorporated herein by reference. Non-limiting techniques for nanotube production include laser vaporization techniques, electric arc techniques, and gas phase techniques.

In still further embodiments, the elongate structure is a biopolymer. In some instances, the biopolymer is a protein. In other instances, the biopolymer is a nucleic acid. In other words, the biopolymer elongate structure may be employed in the same manner as the above-described nanotube or nanowire to pull the first nucleic acid. The biopolymer elongate structure may have any convenient amino acid or nucleotide structure, as desired.

Where methods include pulling the first nucleic acid via an elongate structure, the elongate structure may be attached to the first nucleic acid via any convenient approach. In some cases, a first nucleic acid may be bound (e.g., covalently bound) to an end of the elongate structure. In select cases, an adapter may be associated with one end of the first nucleic acid (e.g., at the 5′ end or the 3′ end). Any convenient adapter may be employed. In select versions, the elongate structure may have a complementary nucleic acid sequence to the adapter associated with the first nucleic acid such that the two molecules may hybridize when placed in proximity to one another, thereby attaching the elongate structure to the first nucleic acid. In an instance where the elongate structure is a nucleic acid, select nucleobases at the end of the elongate structure (e.g., at the 5′ end or the 3′ end) may be complementary to the to the adapter associated with the first nucleic acid such that the two molecules may hybridize when placed in proximity to one another.

In other cases, the force causing movement of the first nucleic acid relative to the second nucleic acid is an electromagnetic force. In such cases, methods include applying a voltage across the opening formed at least in part by the second nucleic acid such that the first nucleic acid moves relative to the second nucleic acid. In some cases, the rate at which the nucleic acid is pulled can be adjusted by adjusting the applied voltage. Voltages for use in the subject methods may vary, and in some instances may range from 25 mV to 500 mV, such as 50 mV to 400 mV, such as 75 mV to 300 mV and including 100 mV to 200 mV. In additional cases, the force causing movement of the first nucleic acid relative to the second nucleic acid is an magnetic force. In some such cases, the first nucleic acid includes a magnetic particle (e.g., magnetic bead) attached thereto. The type of magnetic particle employed may vary, and can include, for example, iron nanoparticles, nickel nanoparticles, cobalt nanoparticles, and the like. In embodiments, applying a magnetic field to the magnetic particle attached to the first nucleic acid is sufficient to provide a pulling force to the first nucleic acid.

The second nucleic acid can be any convenient electrolyzed nucleic acid configured in such a manner that it at least partially forms an opening. In embodiments, the second nucleic acid includes at least a nucleobase that base pairs with adenine, a nucleobase that base pairs with thymine or uracil, a nucleobase that base pairs with guanine, and a nucleobase that base pairs with cytosine. In certain cases, the second nucleic acid includes one or more abasic nucleotides. The second nucleic acid may, in some cases, be a single stranded nucleic acid. As discussed herein, an “electrolyzed” nucleic acid refers to nucleic acid through which an electric current is being passed. Without being bound by theory, it is believed that DNA backbones can support multiple charge transfer mechanisms that arise from the small activation gaps induced by water and counterions. In some embodiments, the second nucleic acid is modified to adjust conductivity. For example, the conductivity of the nucleic acid can be modified with dopants such as conductive metal nanoparticles. Metal nanoparticles of interest include, but are not limited to, gold, lead sulfide, lead selenide, germanium, and silver. In some embodiments, the conductivity of the nucleic acid can be modified with dopants such as conductive carbon, such as carbon nanotubes, carbon nanorods, carbon black, graphene sheets, graphene nanoribbons, and carbon nanofibers. In additional cases, the conductivity of the second nucleic acid is modified by molecular doping with intercalators such as anthraquinone, ferrocene, norbornadiene, methylene blue, ethidium, coralyne and cryptolepin. Nucleic acid intercalation involves the insertion of one intercalating moiety (mono-intercalator), two intercalating moieties (bis-intercalator) or multiple intercalating moieties into the nucleic acid structure. Intercalation changes the conduction/resistance of the complex influencing the overall conduction characteristics of the nucleic acid strand. In one embodiment, the dopant can be reduced or oxidized by the applied current.

In embodiments, the second nucleic acid is attached to a surface. The second nucleic acid may be stably associated with the surface in any suitable manner. By “stably associated”, it is meant that the second nucleic acid does not readily dissociate from the surface. In certain cases, the electrolyzed second nucleic acid comprises first and second discontinuous regions attached to a surface. By “discontinuous” regions, it is meant that the first and second regions are not the same regions (i.e., they do not overlap). The discontinuous regions of the second nucleic acid may be attached to the same or different surfaces. Each surface may be comprised of any convenient material. In certain cases, the surface is a metal surface (e.g., a gold surface). In some embodiments, the second nucleic acid is stably associated with the surface via thiol bonding chemistry. For example, in embodiments where the surface is a gold surface, thiols may directly react with the gold surface to form Au—S bonds via an oxidation-reduction reaction. In additional embodiments, stably associating the second nucleic acid with the surface comprises dip pen nanolithography. In such embodiments, an atomic force microscope may be employed to imprint thiolates onto the surfaces. Dip pen nanolithography is described in, e.g., U.S. Pat. Nos. 8,261,662; 9,403,180; the disclosures of which are herein incorporated by reference in their entirety. In other embodiments, the second nucleic acid is stably associated with the surface via a biotin-streptavidin interaction. In select cases, the second nucleic acid is in contact with multiple surfaces. In other words, one end of the second nucleic acid may be in contact with a first surface, and the other end of the second nucleic acid may be in contact with a second surface that is distinct from the first surface.

In select cases, methods include spatially targeting the attachment of the second nucleic acid by coating the surface with a biocompatible layer. The biocompatible layer may vary and can include, e.g., a polymer matrix, gel, or self-assembled monolayer (SAM). In certain embodiments, an alkanethiol SAM is produced on one or more of the surfaces. In such cases, the surface (e.g., gold surface) is incubated with a thiol-functionalized blocking molecule. Thiol-functionalized blocking molecules include, but are not limited to 1-mercapto-11-undecanol, 1-mercapto-6-hexanol, hexadecanethiol, combinations thereof and the like. SAM formation is described in, e.g., Szymonik et al. Nanotechnology, 27(39), 395301; herein incorporated by reference in its entirety. Methods of interest may additionally include applying a negative voltage to the surface in solution, thereby causing the thiol bond to break and releasing the blocking molecule. Suitable methods for such electrochemical desorption may be found in, e.g., Widrig et al. Journal of electroanalytical chemistry and interfacial electrochemistry, 310(1-2), 335-359; herein incorporated by reference in its entirety. After the release of the blocking molecule, methods according to certain embodiments include incubating a mixture of 5′- or 3′-thiol functionalized nucleic acid (e.g., oligonucleotides) of interest and a thiol-containing blocking molecule to form a new electrode-bound monolayer interspersed with the nucleic acid of interest. Methods that may be adopted for use in surface functionalization are described in, e.g., Wälti et al. Langmuir, 19(4), 981-984; herein incorporated by reference in its entirety.

In some cases, the surface(s) to which the second nucleic acid is bound is an electrode. Put another way, in order to pass the current through the electrolyzed second nucleic acid, the 5′ and/or 3′ end of the nucleic acid may in some cases be in contact with at least one electrode. In select versions, one end of the second nucleic acid is stably associated with an electrode, while the other end is stably associated with a non-electrode surface. In other cases, both ends of the second nucleic acid are stably associated with different electrodes. The electrode(s) may be comprised of any convenient material. In some embodiments, electrodes are metal electrodes. Materials for use in metal electrodes include, but are not limited to platinum, gold, titanium nitride, silver, and graphite. In certain embodiments, the electrodes are gold electrodes.

In select embodiments, the surface (e.g., electrode surface) is functionalized with oligonucleotides comprising sequences complementary to the first and second discontinuous regions and the first and second discontinuous regions are attached to the surface via hybridization to the oligonucleotides. In other words, embodiments of the subject methods include stably associating the second nucleic acid to the surfaces by anchoring an oligonucleotide to one or more of the surfaces (e.g., via thiol bonding chemistry, etc.). The oligonucleotide(s) may be any suitable short (e.g., 5-20 nucleotides) single-stranded DNA or RNA molecule. In certain cases, each oligonucleotide has a sequence that is complementary to a sequence of the second nucleic acid, i.e., such that the two molecules hybridize. Because the oligonucleotides are anchored to a surface (e.g., electrode surface), a second nucleic acid hybridized to the oligonucleotides can be stably associated with the at least one surface.

As discussed above, the second nucleic acid forms at least part of an opening. By “at least part” of an opening, it is meant that the second nucleic acid may make up one component of the opening, or the whole opening. The second nucleic acid may be arranged in any convenient configuration that facilitates sequential interactions between nucleobases of the first nucleic acid and one or more nucleobases of the electrolyzed second nucleic acid. In some instances, methods include arranging the second nucleic acid in a “bridge structure” configuration. As described herein, a bridge structure refers to a configuration of the second nucleic acid in which said nucleic acid is stably associated with a surface (e.g., electrode) in such a manner that a bridge-like shape is formed. In certain instances, the electrolyzed second nucleic acid comprises first and second discontinuous regions attached to a surface or surfaces such that the electrolyzed second nucleic acid forms a bridge structure. In embodiments of the method, movement of the first nucleic acid includes pulling the first nucleic acid through the opening of the bridge structure (e.g., via any of the methods discussed above).

In some cases, the electrolyzed second nucleic acid is one of a plurality of electrolyzed nucleic acids. Any suitable number of electrolyzed nucleic acids may be employed. In some cases, the number of electrolyzed nucleic acids ranges from 1 to 20, such as 1 to 15, and including 1 to 10. In some embodiments, methods of the invention involve the use of 2 or more electrolyzed nucleic acids. In still other embodiments, methods of the invention involve the use of 3 or more electrolyzed nucleic acids. Each of the plurality of electrolyzed nucleic acids may adopt the same configuration, or different configurations. In some embodiments, each of the plurality of electrolyzed nucleic acids is arranged in a bridge structure configuration.

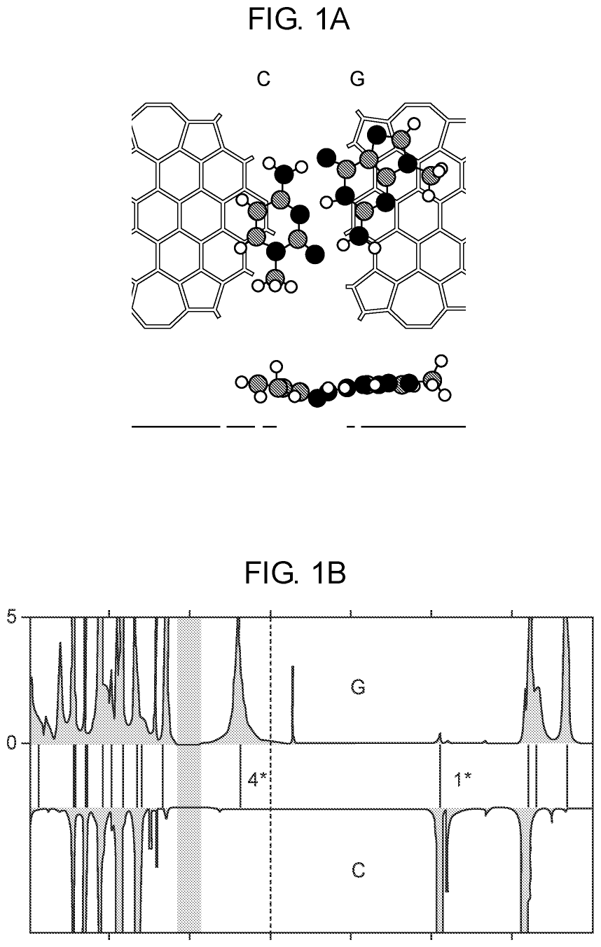

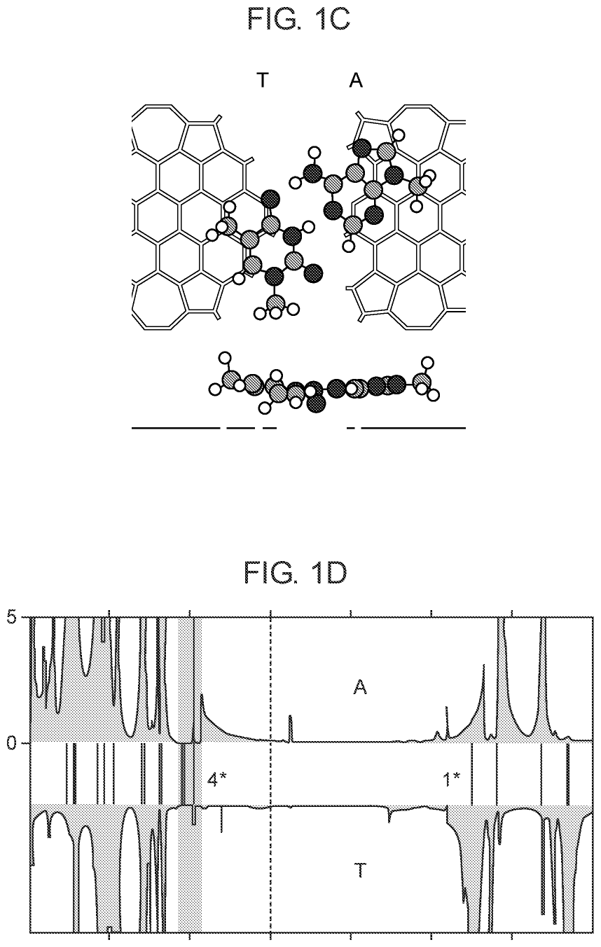

As discussed above, methods of the invention include detecting a varying conductance along the first nucleic acid indicative of sequential interactions between nucleobases of the first nucleic acid and one or more nucleobases of the electrolyzed second nucleic acid. As discussed above in the Summary section with respect to Agapito et al. and FIG. 1, interactions between given nucleobases of nucleic acids are characterized by a particular conductance fingerprint. In other words, the DNA base pair behaves as a biological Aviram-Ratner electrical rectifier because of the spatial separation and weak bonding between the nucleobases, and because the current that flow across these base pairs varies based on the specific nature of the bond. As such, methods include identifying these conductance fingerprints as the first nucleic acid passes through the opening formed at least in part by the second nucleic acid, and thereby identifying characteristics of the first nucleic acid including, but not limited to, the sequence of the first nucleic acid. As such, methods include applying an electrical current to the second nucleic acid (e.g., via an electrode) in a manner sufficient for the current to pass through the backbone of the second nucleic acid.

By detecting a varying conductance “along” the first nucleic acid, it is meant that the current travels across the electrolyzed second nucleic acid (e.g., bridge structure) to the DNA/RNA/molecule to be sequenced (i.e., the first nucleic acid). As such, in these reiterations the electrons travel along the first nucleic acid to complete the circuit. The interaction between the base to be sequenced and the second nucleic acid bases will lead to a varying conductance fingerprint which will allow for the identification of the sequenced base. Measurement of the electrons traveling along the first nucleic acid allows for the assessment of said conductance fingerprint.

The conductance along the first nucleic acid may be measured via any convenient approach. In some embodiments, an electrometer is employed to provide a bias current and analyze the resultant current. Commercially available electrometers that may be suitable for use in the subject methods include, e.g., Keithley® instruments. In some embodiments, detecting the varying conductance includes an impedance-based approach. In certain versions, methods include identifying a nucleobase based its characteristic energy levels. Characteristic energy levels of interest include, but are not limited to, highest occupied molecular orbital (HOMO) energy. Measurement techniques that may be adapted for use in the subject methods can be found in, e.g., Pedersen et al. Nanotechnology, 28(1), 015502; and Ohshiro et al. 2012 12th IEEE International Conference on Nanotechnology (IEEE-NANO) (pp. 1-2). IEEE; herein incorporated by reference in their entirety.

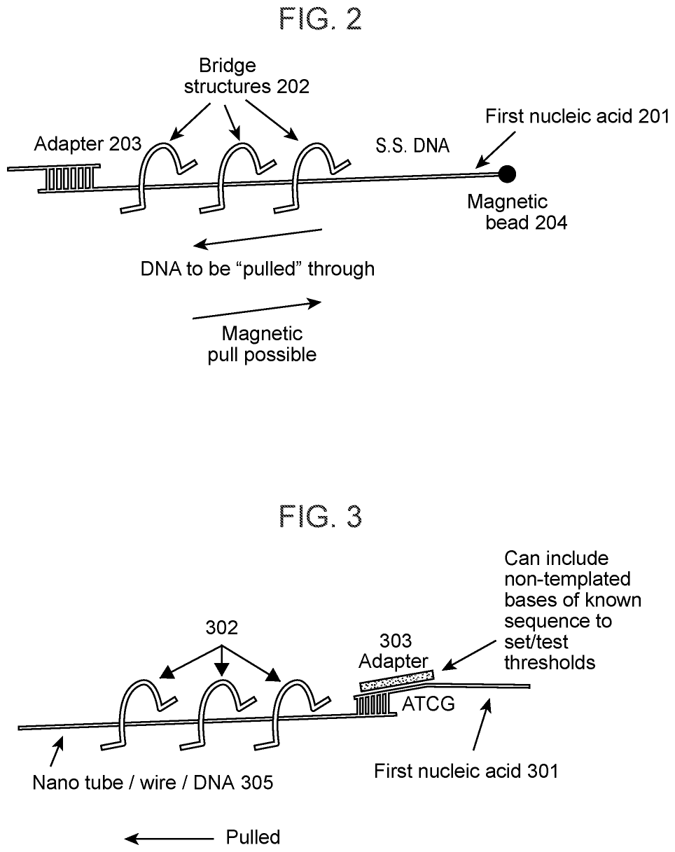

FIG. 2 presents a schematic illustration of a method according to embodiments of the present disclosure comprising relative movement of a first nucleic acid through a plurality of openings formed by a plurality of electrolyzed nucleic acids. In this example, the electrolyzed second nucleic acids 202 each comprise first and second discontinuous regions attached to a surface such that the electrolyzed nucleic acids 202 form bridge structures. During the relative movement, a varying conductance is detected along the first nucleic acid 201 indicative of sequential interactions between nucleobases of the first nucleic acid 201 and one or more nucleobases of the electrolyzed bridge structures 202. In this example, the relative movement comprises pulling the first nucleic acid through the openings formed by the bridge structures 202. An adapter 203 is hybridized to the first nucleic acid 201 that connects the first nucleic acid 201 to an elongate structure (not shown) for pulling the first nucleic acid 201 through the bridge structures 202. In the embodiment of FIG. 2, first nucleic acid 201 is attached to magnetic bead 204.

FIG. 3 presents a schematic illustration of a method according to embodiments of the present disclosure comprising relative movement of a first nucleic acid through a plurality of openings formed by a plurality of electrolyzed nucleic acids. In this example, the electrolyzed second nucleic acids 302 each comprise first and second discontinuous regions attached to a surface such that the electrolyzed nucleic acids form bridge structures. During the relative movement, a varying conductance is detected along the first nucleic acid 301 indicative of sequential interactions between nucleobases of the first nucleic acid 301 and one or more nucleobases of the electrolyzed bridge structures 302. In this example, the relative movement comprises pulling the first nucleic acid through the openings formed by the bridge structures, wherein the pulling is via an elongate structure 305 (e.g., a nanotube, nanowire, or biopolymer (e.g., nucleic acid)) in the direction that the first nucleic acid 301 is to be pulled. The elongate structure 305 is attached to the first nucleic acid 301 via adapter 303.

In embodiments, causing the relative movement comprises moving the surface relative to the first nucleic acid. In other words, the relative movement is caused by movement of the surface and the movement of the first nucleic acid is negligible. In certain instances the first nucleic acid is immobilized during the relative movement. Any convenient approach may be employed to move the surface, such as employing piezoelectric materials/actuators. In some embodiments, the materials and approaches using in mechanically controllable break junctions and AFM may be employed to move the surface.

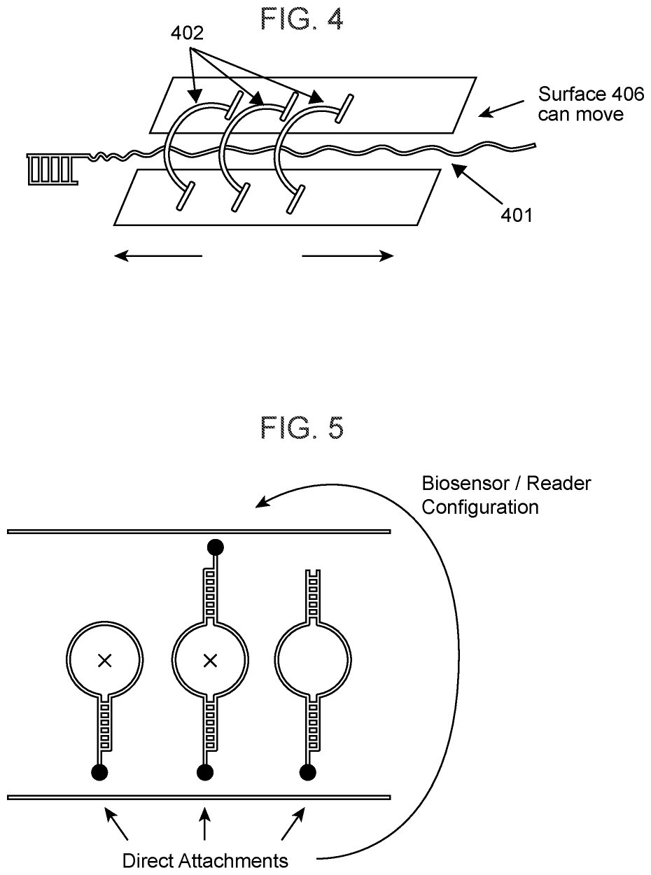

FIG. 4 presents an illustration of a method according to embodiments of the present disclosure comprising relative movement of a first nucleic acid through a plurality of openings formed by a plurality of electrolyzed nucleic acids. In this example, the electrolyzed nucleic acids each comprise first and second discontinuous regions attached to a surface 406 such that the electrolyzed nucleic acids form bridge structures 402. During the relative movement, a varying conductance is detected along the first nucleic acid 401 indicative of sequential interactions between nucleobases of the first nucleic acid 401 and one or more nucleobases of the electrolyzed bridge structures 402. In this example, the relative movement comprises moving the surface 406 (and in turn, the bridge structures 402) relative to the first nucleic acid 401.

In certain versions of the disclosed methods, the electrolyzed second nucleic acid at least in part forms a loop structure, and the relative movement of the first nucleic acid is through the opening of the loop structure. For example, the electrolyzed second nucleic acid may include a first end attached to a surface (e.g., an electrode) and form a stem-loop structure. In additional embodiments, the electrolyzed second nucleic acid comprises a first end attached to a surface and first and second discontinuous regions hybridized to a third nucleic acid molecule such that the electrolyzed second nucleic acid and third nucleic acid molecule form a loop structure. The third nucleic acid may be substantially similar to the second nucleic acid described herein. In select instances, the third nucleic acid is also electrolyzed. In certain cases, the third nucleic acid possesses one or more regions that are complementary to regions of the second nucleic acid such that the two nucleic acids may hybridize and thereby form a loop structure. In select cases, the second nucleic acid is attached to a first surface, and the third nucleic acid is attached to a second surface. In other cases, only one of the second and third nucleic acids is attached to a surface. The second and/or third nucleic acids may be attached to the substrate(s) via any convenient mechanism, such as those described above. In select embodiments, the first end of the electrolyzed second nucleic acid is attached to the surface via a biotin-streptavidin interaction. In additional embodiments, the first end of the electrolyzed second nucleic acid is attached to the surface via magnetic attraction.

FIG. 5 presents a schematic illustration of various electrolyzed nucleic acid configurations that find use in practicing the methods of the present disclosure. As shown on the left, the electrolyzed second nucleic acid may comprise a first end attached to a surface and forms a stem-loop structure. As shown in the middle, an electrolyzed second nucleic acid may comprise a first end attached to a surface and first and second discontinuous regions hybridized to a third nucleic acid molecule such that the electrolyzed second nucleic acid and third nucleic acid molecule form a loop structure. In this example, the third nucleic acid comprises an end attached to a second surface. As shown on the right, an electrolyzed second nucleic acid may comprise a first end attached to a surface and first and second discontinuous regions hybridized to a third nucleic acid molecule such that the electrolyzed second nucleic acid and third nucleic acid molecule form a loop structure. In this example, the third nucleic acid does not comprise an end attached to a second surface. The DNA to be sequenced (i.e., the first nucleic acid, marked by an “X”) is internal to the loop, and the concept and method of completing the circuit is identical to that in FIGS. 1-4.

With reference to FIG. 5, in some embodiments, the structures shown (e.g., the middle structure attached to each substrate) are disposed in such a manner that the loop(s) form(s) a nanopore used for sequencing the first nucleic acid. For example, the DNA that forms the hairpin may have a region that is conductive and a region that acts as an isolator.

In some cases, methods include detecting a varying conductance between the first nucleic acid and an electrode proximate to the first nucleic acid. As with the embodiments discussed above with respect to FIGS. 1-5, the varying conductance is indicative of sequential interactions between nucleobases of the first nucleic acid and one or more nucleobases of the electrolyzed second nucleic acid and the current flows along the DNA molecule to be sequenced. However, without being bound by theory, the circuit could also be completed by placing a conductive electrode in the proximity of the second nucleic acid and the nucleic acid to be sequenced (i.e., the first nucleic acid). The interaction between the base to be sequenced and the second nucleic acid bases will lead to a varying conductance fingerprint which will allow for the identification of the sequenced base. The electrode proximate to the first nucleic acid may be any convenient electrode, such as those described above.

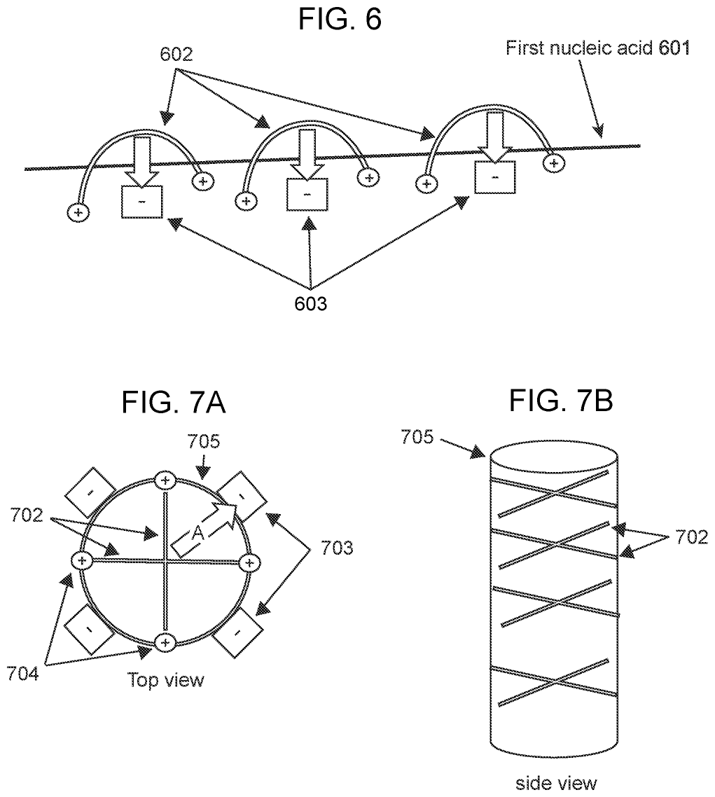

FIG. 6 presents a schematic illustration of embodiments in which a varying conductance between the first nucleic acid and an electrode proximate to the first nucleic acid is detected, wherein the varying conductance is indicative of sequential interactions between nucleobases of the first nucleic acid and one or more nucleobases of electrolyzed nucleic acids in the form of bridge structures. In the example of FIG. 6, relative movement occurs between first nucleic acid 601 and plurality of (i.e., 3) electrolyzed second nucleic acids 602 (e.g., via any one of the mechanisms described above). Current from the plurality of electrolyzed nucleic acids 602 passes through first nucleic acid 601 as base-base interactions occur between the nucleic acids, and travels to electrodes 603 in the direction of the arrows. Conductance may subsequently be evaluated for the above-described fingerprints to identify the nucleobases of the first nucleic acid 601.

In some cases, the electrolyzed second nucleic acid is disposed within a channel, and causing the relative movement between the first and second nucleic acid includes translocating the first nucleic acid through the opening formed at least in part by the electrolyzed second nucleic acid within the channel. Any channel suitable for translocating a nucleic acid may be employed. The size (e.g., diameter) of the channel may vary. Exemplary diameters range from 0.5 nm to 20 nm. Materials from which the channels may be constructed include, but are not limited to, silicon (e.g., silicon nitride), graphene, or the like, and combinations thereof. In some cases, the channel is a nanopore (e.g., a nanopore across which a potential difference is applied), and methods include exposing the nucleobases to the nanopore in a sequential manner while monitoring for electrical signals.

The second nucleic acid may be arranged within the channel in any convenient manner. In some embodiments, the second nucleic acid is attached at one end to the channel at a first point, and attached at the other end to the channel at a second point. In some cases, the second point is opposite the first point. The second nucleic acid may be attached to the channel by any convenient technique, including but not limited to the techniques described above (e.g., thiol-based techniques). In certain embodiments, the second nucleic acid is one of a plurality of electrolyzed nucleic acids arranged within the channel. In these embodiments, the electrolyzed nucleic acids may be arranged with respect to each other in any convenient manner. In select versions, the electrolyzed nucleic acids are arranged in a “crosshairs” configuration. In other words, the electrolyzed nucleic acids are attached to the pore such that the resulting shape of the electrolyzed nucleic acids resembles a cross from the vantage point of the top of the pore. The electrolyzed nucleic acids may be attached to electrodes (e.g., positive electrodes) within the channel which are configured to apply a current therethrough. The crosshairs configuration results in the creation of 4 quadrants within the channel, any one of which the first nucleic acid may pass through. In embodiments, each quadrant is associated with an electrode (e.g., negative electrode). Current from the electrolyzed nucleic acids can be conducted through the first nucleic acid that is passing through a given quadrant before it reaches the respective negative electrode. Varying conductance between the first nucleic acid and the electrode proximate to the first nucleic acid can then be employed to identify characteristics of the first nucleic acid (e.g., sequence information).

Any nanopore device/apparatus suitable for translocating a first nucleic acid therethrough and detecting/monitoring varying conductance during the translocating may be employed when practicing the subject methods. For example, a suitable nanopore device may include a chamber including an aqueous solution and a membrane that separates the chamber into two sections, the membrane including a nanopore formed therein. Electrical measurements may be made using single channel recording equipment such as that described, e.g., in Lieberman et al. (2010) J. Am. Chem. Soc. 132(50):17961-72; Stoddart et al. (2009) PNAS 106(19):7702-7; U.S. Pat. No. 9,481,908; and U.S. Patent Application Publication No. US2014/0051068; the disclosures of which are incorporated herein by reference in their entireties for all purposes. Alternatively, electrical measurements may be made using a multi-channel system, for example as described in U.S. Patent Application Publication No. US2015346149, the disclosure of which is incorporated herein by reference in its entirety for all purposes.

In nanopore-based analysis (e.g., sequencing), the nanopore serves as a biosensor and provides the sole passage through which an ionic solution on the cis side of the membrane contacts the ionic solution on the trans side. A constant voltage bias (trans side positive) produces an ionic current through the nanopore and drives polynucleotides in the cis chamber through the pore to the trans chamber. A processive enzyme (e.g., a helicase, polymerase, nuclease, or the like) may be bound to the polynucleotide such that its step-wise movement controls and ratchets the nucleotides through the small-diameter nanopore, nucleobase by nucleobase.

Suitable conditions for nanopore-based analysis (e.g., protein pores, solid state pores, etc.) are known in the art. Typically, a voltage is applied across the membrane and pore. The voltage used may be from +2 V to −2 V, e.g., from −400 mV to +400 mV. The voltage used may be in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage may be in the range of from 100 mV to 240 mV, e.g., from 120 mV to 220 mV.

The methods are typically carried out in the presence of a suitable charge carrier, such as metal salts, for example alkali metal salts, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or I-ethyl-3-methyl imidazolium chloride. Generally, the salt is present in the aqueous solution in the chamber. Potassium chloride (KCl), sodium chloride (NaCl) or cesium chloride (CsCl) may be used, for example. The salt concentration may be at saturation. The salt concentration may be 3M or lower and is typically from 0.1 to 2.5 M, from 0.3 to 1.9 M, from 0.5 to 1.8 M, from 0.7 to 1.7 M, from 0.9 to 1.6 M, or from 1 M to 1.4 M. The salt concentration may be from 150 mM to 1 M. The methods are preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

In some embodiments, the rate at which the first nucleic acid is exposed to the nanopore is controlled using a processive enzyme. Non-limiting examples of processive enzymes that may be employed include polymerases (e.g., a phi29 or other suitable polymerase) and helicases, e.g., a Hel308 helicase, a RecD helicase, a Tral helicase, a Tral subgroup helicase, an XPD helicase, or the like. The processive enzyme may bind, e.g., the nucleic acid, followed by the resulting complex being drawn to the nanopore, e.g., by a potential difference applied across the nanopore. In other embodiments, the processive enzyme may be located at the nanopore (e.g., attached to or adjacent to the nanopore) such that the processive enzyme binds, e.g., the nucleic acid upon arrival at the nanopore.

The nanopore may be present in a solid-state film, a biological membrane, or the like. In some embodiments, the nanopore is a solid-state nanopore. In other embodiments, the nanopore is a biological nanopore. The biological nanopore may be, e.g., an alpha-hemolysin-based nanopore, a Mycobacterium smegmatis porin A (MspA)-based nanopore, or the like.

FIG. 7A-7B present embodiments of the invention in which the electrolyzed second nucleic acid is disposed within a channel. FIG. 7A presents a top view of channel 705, while FIG. 7B presents a side view of channel 705. As shown in FIG. 7A, channel (i.e., nanopore) 705 includes negative electrodes 703 and positive electrodes 704. Electrolyzed nucleic acids 702 are attached to channel 705 via electrodes 704, and are arranged in a crosshairs configuration which results in a division of the interior of channel 705 into 4 quadrants. A nucleic acid to be sequenced (i.e., a first amino acid) can pass through any one of the 4 quadrants. The molecule to be sequenced can be pulled through the pore and through the bridge structure via electrophoresis, or via the flow of a fluid/buffer through the pore. Illustrated is the base “A” allowing the circuit to be completed. FIG. 7B presents an embodiment of channel 705 in which multiple sets of electrolyzed nucleic acids 702. While FIG. 7B does not depict the electrodes shown in FIG. 7A, their presence is implied.

In embodiments, the subject methods are carried out on an integrated device configured to execute the steps of the present invention. The integrated device may be configured to analyze (e.g., sequence) one or more nucleic acids (i.e., first nucleic acids) using one or more electrolyzed nucleic acids (i.e., second nucleic acids) described herein. The integrated device may include components necessary to electrolyze the second nucleic acid, such as power sources, electrodes, electrical conduits, and switches. The integrated device may be configured to detect a varying conductance along the first nucleic acid, or detect a varying conductance between the first nucleic acid and an electrode proximate to the first nucleic acid, as desired. In select embodiments, the integrated device is a chip. The chip may be constructed from any convenient material. An exemplary material includes silicon (e.g., silicon dioxide).

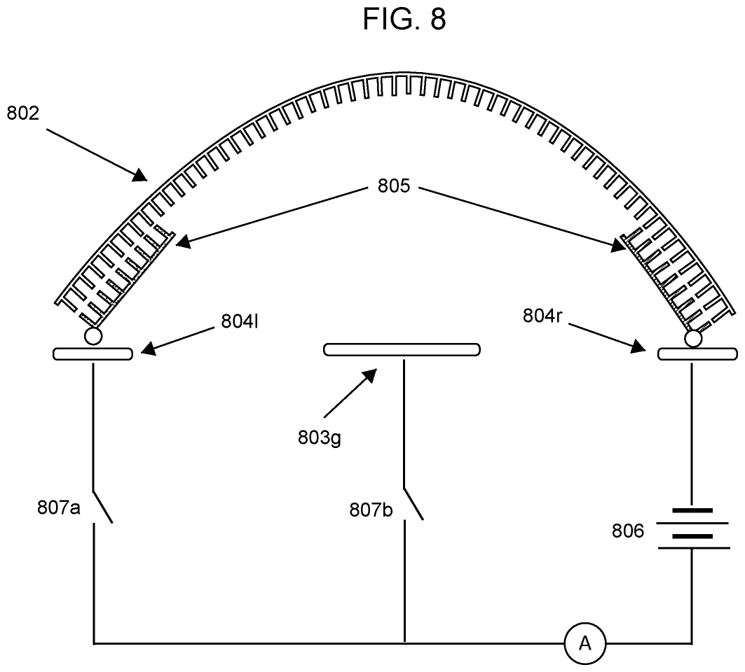

FIG. 8 depicts an embodiment of the invention in which a varying conductance can be detected between a first nucleic acid and an electrode proximate to the first nucleic acid. As shown in FIG. 8, oligonucleotides are attached to electrodes 804l on the left and 804r on the right (e.g., via thiol chemistry, as described above). Second nucleic acid 802 hybridizes to oligonucleotides 805, thereby forming an opening through which a nucleic acid to be sequenced may pass. Current originating from power source 806 (e.g., battery) can be applied to one or both of the electrodes 804l (left electrode) and 804r (right electrode). For example, depending on whether switch 807a disengaged, current may or may not be applied to the electrode 804l. When second nucleic acid 802 is electrolyzed, current may jump from second nucleic acid 802 to electrode 803g. When a nucleic acid to be sequenced passes through the opening formed by nucleic acid 802, base-base interactions between the two nucleic acids leads to the presence of conductance fingerprints that may be detected by the device. A circuit may be completed depending on whether switch 807b is engaged or disengaged.

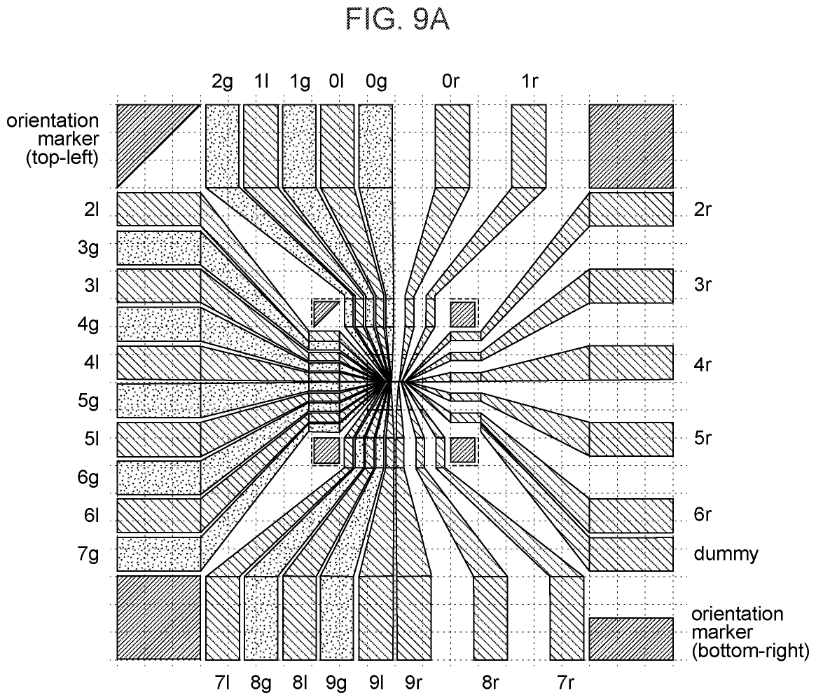

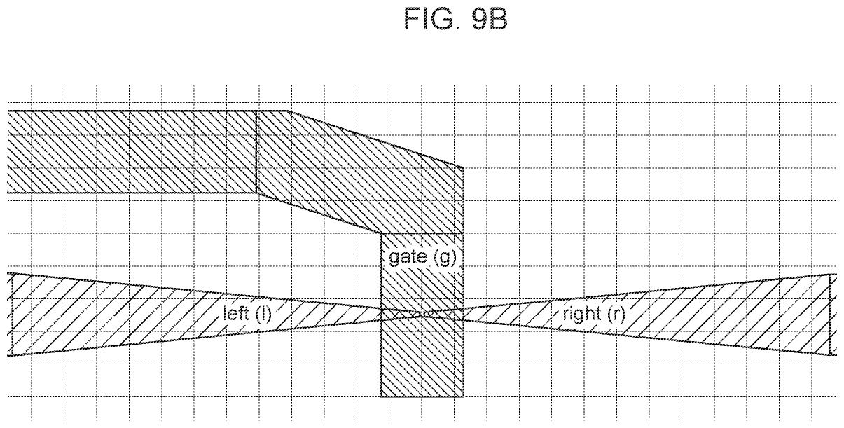

FIG. 9A-9B depict the architecture of a chip for use in the subject methods. As shown in FIG. 9A, the chip includes a plurality of (numbered 0-7) left and right electrodes (denoted by “l” and “r”, respectively) corresponding to electrodes 804l and 804r shown in FIG. 8. In addition, the chip includes “gate” electrodes corresponding to electrode 803g in FIG. 8. As shown in FIG. 9B, each of the left and right electrodes are separated by a small gap (e.g., ranging in length from 20 nm to 60 nm) across which the second nucleic acid is positioned (e.g., as shown in FIG. 8). Under the left and right electrodes is the gate electrode, which is insulated from said left and right electrodes.

According to some embodiments, the methods of the present disclosure are computer-implemented. By “computer-implemented” is meant at least one step of the method is implemented using one or more processors and one or more non-transitory computer-readable media. The computer-implemented methods of the present disclosure may further comprise one or more steps that are not computer-implemented, e.g., obtaining a sample from a subject, isolating nucleic acids for sequencing, performing a contacting and/or combining step according to the methods of the present disclosure, and/or the like.

A nucleic acid to be sequenced according to the methods of the present disclosure may be a deoxyribonucleic acid (DNA). DNAs of interest include, but are not limited to, genomic DNA or fragments thereof, complementary DNA (or “cDNA”, synthesized from any RNA or DNA of interest) or fragments thereof, recombinant DNA (e.g., plasmid DNA) or fragments thereof, and/or the like. The nucleic acid to be sequenced may be greater than about 2 bases, greater than about 10 bases, greater than about 100 bases, greater than about 500 bases, greater than 1000 bases, greater than 10,000 bases, greater than 100,000 bases, greater than about 1,000,000, up to about 10¹⁰ or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

A nucleic acid to be sequenced according to the methods of the present disclosure may be a ribonucleic acid (RNA). The RNA may be any type of RNA (or sub-type thereof) including, but not limited to, a messenger RNA (mRNA), a microRNA (miRNA), a small interfering RNA (siRNA), a transacting small interfering RNA (ta-siRNA), a natural small interfering RNA (nat-siRNA), a ribosomal RNA (rRNA), a transfer RNA (tRNA), a small nucleolar RNA (snoRNA), a small nuclear RNA (snRNA), a long non-coding RNA (lncRNA), a non-coding RNA (ncRNA), a transfer-messenger RNA (tmRNA), a precursor messenger RNA (pre-mRNA), a small Cajal body-specific RNA (scaRNA), a piwi-interacting RNA (piRNA), an endoribonuclease-prepared siRNA (esiRNA), a small temporal RNA (stRNA), a signal recognition RNA, a telomere RNA, a ribozyme, or any combination of RNA types thereof or subtypes thereof.

In certain embodiments, moieties of the nucleic acid to be sequenced comprise a “non-natural nucleoside” or “non-natural nucleotide”, which refer to a nucleoside or nucleotide that contains a modified nucleobase and/or other chemical modification, such as a modified sugar. In some cases, non-natural nucleotides/nucleosides possess a unique conductance fingerprint that may be recognized by the subject methods. According to some embodiments, the molecular disks comprise moieties that comprise non-natural nucleobases and/or non-natural nucleotides that modify the melting temperature (Tm) of a synthetic strand-nucleic acid hybrid as compared to a nucleic acid-nucleic acid hybrid. Non-limiting examples include modified pyrimidine such as methyl-dC or propynyl-dU; modified purine, e.g., G-clamp; 2-Amino-2′-deoxyadenosine-5′-Triphosphate (2-Amino-dATP), 5-Methyl-2′-deoxycytidine-5′-Triphosphate (5-Me-dCTP), 5-Propynyl-2′-deoxycytidine-5′-Triphosphate (5-Pr-dCTP), 5-Propynyl-2′-deoxyuridine-5′-Triphosphate (5-Pr-dUTP), a halogenated deoxy-uridine (XdU) such as 5-Chloro-2′-deoxyuridine-5′-Triphosphate (5-Cl-dUTP), 5-Bromo-2′-deoxyuridine-5′-Triphosphate (5-Br-dUTP), or any combination thereof.

A nucleic acid to be sequenced according to the methods of the present disclosure may be a nucleic acid from one or more immune cells. Immune cells of interest include, but are not limited to, T cells, B cells, natural killer (NK) cells, macrophages, monocytes, neutrophils, dendritic cells, mast cells, basophils, and eosinophils. In certain embodiments, the nucleic acid to be sequenced is from a T cell. T cells of interest include naive T cells (T_{N}), cytotoxic T cells (T_{CTL}), memory T cells (T_{MEM}), T memory stem cells (T_{SCM}), central memory T cells (T_{CM}), effector memory T cells (T_{EM}), tissue resident memory T cells (T_{RM}), effector T cells (T_{EFF}), regulatory T cells (T_{REGs}), helper T cells (T_{H}, T_{H}1, T_{H}2, T_{H}17) CD4+ T cells, CD8+ T cells, virus-specific T cells, alpha beta T cells (T_{αβ}), and gamma delta T cells (T_{γδ}).

In certain embodiments, a nucleic acid to be sequenced according to the methods of the present disclosure is a nucleic that encodes an immune cell receptor (e.g., a T cell receptor (TCR), a B cell receptor (BCR)) or a portion thereof. For example, in certain embodiments, provided are methods that comprise sequencing a nucleic acid that encodes one or more CDRs of an alpha chain or a beta chain of a TCR. According to some embodiments, the methods comprise sequencing a CDR3-encoding portion of a nucleic acid that encodes all or a portion of an alpha chain or a beta chain of a TCR. In certain embodiments, such methods employ a synthetic strand comprising a series of molecular disks each comprising a moiety for binding to A, C, G, or T/U, where each molecular disk of the series binds exclusively to A, C, G, or T/U, and where the series is designed to hybridize to a known nucleotide sequence (e.g., a constant region sequence) adjacent the CDR3-encoding portion of the nucleic acid that encodes all or a portion of an alpha chain or a beta chain of a TCR, such that the nucleotide sequence of the CDR3-encoding portion may be determined based on the rotational positions of the molecular disks adjacent the series of molecular disks.

The nucleic acids to be sequenced according to the methods of the present disclosure may be present in any nucleic acid sample of interest. In certain embodiments, the nucleic acids are present in a nucleic acid sample isolated from a single cell, a plurality of cells (e.g., cultured cells), a tissue, an organ, or an organism (e.g., bacteria, yeast, or the like). According to some embodiments, the nucleic acid sample is isolated from a cell(s), tissue, organ, and/or the like of an animal. In some embodiments, the animal is a mammal, e.g., a mammal from the genus Homo (e.g., a human), a rodent (e.g., a mouse or rat), a dog, a cat, a horse, a cow, or any other mammal of interest. In certain embodiments, the nucleic acid sample is isolated/obtained from a source other than a mammal, such as bacteria, yeast, insects (e.g., drosophila), amphibians (e.g., frogs (e.g., Xenopus)), viruses, plants, or any other non-mammalian nucleic acid sample source.

Nucleic acids that may be sequenced according to the methods of the present disclosure include cell-free nucleic acids, e.g., cell-free DNA, cell-free RNA, or both. Such cell-free nucleic acids may be obtained from any suitable source. In certain embodiments, the cell-free nucleic acids are from a body fluid sample selected from the group consisting of: whole blood, blood plasma, blood serum, amniotic fluid, saliva, urine, pleural effusion, bronchial lavage, bronchial aspirates, breast milk, colostrum, tears, seminal fluid, peritoneal fluid, pleural effusion, and stool. In certain embodiments, the cell-free nucleic acids are cell-free fetal DNAs. According to some embodiments, the cell-free nucleic acids are circulating tumor DNAs. In certain embodiments, the cell-free nucleic acids comprise infectious agent DNAs. According to some embodiments, the cell-free nucleic acids comprise DNAs from a transplant.

The term “cell-free nucleic acid” as used herein can refer to nucleic acid isolated from a source having substantially no cells. Cell-free nucleic acid may be referred to as “extracellular” nucleic acid, “circulating cell-free” nucleic acid (e.g., CCF fragments, ccf DNA) and/or “cell-free circulating” nucleic acid. Cell-free nucleic acid can be present in and obtained from blood (e.g., from the blood of an animal, from the blood of a human subject). Cell-free nucleic acid often includes no detectable cells and may contain cellular elements or cellular remnants. Non-limiting examples of acellular sources for cell-free nucleic acid are described above. Obtaining cell-free nucleic acid may include obtaining a sample directly (e.g., collecting a sample, e.g., a test sample) or obtaining a sample from another who has collected a sample. According to some embodiments, a cell-free nucleic acid may be a product of cell apoptosis and cell breakdown, which provides basis for cell-free nucleic acid often having a series of lengths across a spectrum (e.g., a “ladder”). In some embodiments, sample nucleic acid from a test subject is circulating cell-free nucleic acid. In some embodiments, circulating cell free nucleic acid is from blood plasma or blood serum from a test subject.

Cell-free nucleic acid can include different nucleic acid species, and therefore is referred to herein as “heterogeneous” in certain embodiments. For example, a sample from a subject having cancer can include nucleic acid from cancer cells (e.g., tumor, neoplasia) and nucleic acid from non-cancer cells. In another example, a sample from a pregnant female can include maternal nucleic acid and fetal nucleic acid. In another example, a sample from a subject having an infection or infectious disease can include host nucleic acid and nucleic acid from the infectious agent (e.g., bacteria, fungus, protozoa). In another example, a sample from a subject having received a transplant can include host nucleic acid and nucleic acid from the donor organ or tissue. In some instances, cancer, fetal, infectious agent, or transplant nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is cancer, fetal, infectious agent, or transplant nucleic acid). In another example, heterogeneous cell-free nucleic acid may include nucleic acid from two or more subjects.

Nucleic acids that may be sequenced according to the methods of the present disclosure include tumor nucleic acids (e.g., present in a nucleic acid sample isolated from a tumor—e.g., a tumor biopsy sample). “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like.

Approaches, reagents and kits for isolating, purifying and/or concentrating DNA and RNA from sources of interest are known in the art and commercially available. For example, kits for isolating DNA from a source of interest include the DNeasy®, RNeasy®, QIAamp®, QIAprep® and QIAquick® nucleic acid isolation/purification kits by Qiagen, Inc. (Germantown, Md); the DNAzol®, ChargeSwitch®, Purelink®, GeneCatcher® nucleic acid isolation/purification kits by Life Technologies, Inc. (Carlsbad, CA); the NucleoMag®, NucleoSpin®, and NucleoBond® nucleic acid isolation/purification kits by Clontech Laboratories, Inc. (Mountain View, CA). In certain embodiments, the nucleic acid is isolated from a fixed biological sample, e.g., formalin-fixed, paraffin-embedded (FFPE) tissue. Genomic DNA from FFPE tissue may be isolated using commercially available kits—such as the Allrep® DNA/RNA FFPE kit by Qiagen, Inc. (Germantown, Md), the RecoverAll® Total Nucleic Acid Isolation kit for FFPE by Life Technologies, Inc. (Carlsbad, CA), and the NucleoSpin® FFPE kits by Clontech Laboratories, Inc. (Mountain View, CA).

Nucleic acid sequences determined according to the methods of the present disclosure may be analyzed (e.g., assembled and/or the like) using available sequence analysis software.

In select embodiments, rather than sequencing a nucleic acid, methods of the invention include sequencing a polypeptide. Amino acids may possess conductance fingerprints in a manner similar to nucleotides. As such, encompassed by the present disclosure are embodiments in which the molecule to be sequenced is a polypeptide. For example, methods of the invention may include causing relative movement of a first polypeptide through an opening formed at least in part by an electrolyzed second polypeptide; and during the relative movement, detecting a varying conductance along the first polypeptide. Alternatively, during the relative movement, methods can include detecting a varying conductance between the first polypeptide and an electrode proximate to the first polypeptide.

The polypeptide to be sequenced may be any polypeptide, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The term “amino acid” generally refers to any monomer unit that comprises a substituted or unsubstituted amino group, a substituted or unsubstituted carboxy group, and one or more side chains or groups, or analogs of any of these groups. Exemplary side chains include, e.g., thiol, seleno, sulfonyl, alkyl, aryl, acyl, keto, azido, hydroxyl, hydrazine, cyano, halo, hydrazide, alkenyl, alkynl, ether, borate, boronate, phospho, phosphono, phosphine, heterocyclic, enone, imine, aldehyde, ester, thioacid, hydroxylamine, or any combination of these groups. Other representative amino acids include, but are not limited to, amino acids comprising photoactivatable cross-linkers, metal binding amino acids, spin-labeled amino acids, fluorescent amino acids, metal-containing amino acids, amino acids with novel functional groups, amino acids that covalently or noncovalently interact with other molecules, photocaged and/or photoisomerizable amino acids, radioactive amino acids, amino acids comprising biotin or a biotin analog, glycosylated amino acids, other carbohydrate modified amino acids, amino acids comprising polyethylene glycol or polyether, heavy atom substituted amino acids, chemically cleavable and/or photocleavable amino acids, carbon-linked sugar-containing amino acids, redox-active amino acids, amino thioacid containing amino acids, and amino acids comprising one or more toxic moieties.

The term “amino acid” includes, but is not limited to, naturally-occurring α-amino acids and their stereoisomers. “Stereoisomers” of amino acids refer to mirror image isomers of the amino acids, such as L-amino acids or D-amino acids. For example, a stereoisomer of a naturally-occurring amino acid refers to the mirror image isomer of the naturally-occurring amino acid (i.e., the D-amino acid).

Naturally-occurring α-amino acids are those encoded by the genetic code as well as those amino acids that are later modified (e.g., hydroxyproline, γ-carboxyglutamate, and O-phosphoserine). Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

### Computer-Readable Media and Systems

Aspects of the present disclosure further include systems, e.g., nucleic acid sequencing systems. In certain embodiments, such systems comprise one or more processors, and one or more non-transitory computer-readable media comprising instructions stored thereon that cause the system to: monitor a varying conductance along a first nucleic acid or a varying conductance between the first nucleic acid and an electrode proximate to the first nucleic acid. The varying conductance monitored by the system is indicative of sequential interactions between nucleobases of a first nucleic acid and one or more nucleobases of an electrolyzed second nucleic acid. In some embodiments, the varying conductance comprises conductance fingerprints for the different nucleobases in the first nucleic acid, and the one or more non-transitory computer-readable media comprises instructions stored thereon that cause the system to determine the identity of one or more nucleotides of the first nucleic acid based on the varying conductance. In additional embodiments, the varying conductance comprises conductance fingerprints for the different nucleobases in the first nucleic acid, and the one or more non-transitory computer-readable media comprises instructions stored thereon that cause the system to determine a nucleotide sequence of the first nucleic acid based on the varying conductance.

A variety of processor-based systems may be employed to implement the embodiments of the present disclosure. Such systems may include system architecture wherein the components of the system are in electrical communication with each other using a bus. System architecture can include a processing unit (CPU or processor), as well as a cache, that are variously coupled to the system bus. The bus couples various system components including system memory, (e.g., read only memory (ROM) and random access memory (RAM), to the processor.

System architecture can include a cache of high-speed memory connected directly with, in close proximity to, or integrated as part of the processor. System architecture can copy data from the memory and/or the storage device to the cache for quick access by the processor. In this way, the cache can provide a performance boost that avoids processor delays while waiting for data. These and other modules can control or be configured to control the processor to perform various actions. Other system memory may be available for use as well. Memory can include multiple different types of memory with different performance characteristics. Processor can include any general purpose processor and a hardware module or software module, such as first, second and third modules stored in the storage device, configured to control the processor as well as a special-purpose processor where software instructions are incorporated into the actual processor design. The processor may essentially be a completely self-contained computing system, containing multiple cores or processors, a bus, memory controller, cache, etc. A multi-core processor may be symmetric or asymmetric.

Aspects of the invention also include non-transitory computer-readable media. The subject non-transitory computer-readable media include instructions stored thereon that cause a system to monitor a varying conductance along a first nucleic acid, or a varying conductance between the first nucleic acid and an electrode proximate to the first nucleic acid. As above, the varying conductance is indicative of sequential interactions between nucleobases of a first nucleic acid and one or more nucleobases of an electrolyzed second nucleic acid. In select cases, the varying conductance comprises conductance fingerprints for the different nucleobases in the first nucleic acid, and the one or more non-transitory computer-readable media comprises instructions stored thereon that cause the system to determine the identity of one or more nucleotides of the first nucleic acid based on the varying conductance. In additional cases, the varying conductance comprises conductance fingerprints for the different nucleobases in the first nucleic acid, and the one or more non-transitory computer-readable media comprises instructions stored thereon that cause the system to determine a nucleotide sequence of the first nucleic acid based on the varying conductance.

To enable user interaction with the computing system architecture, an input device can represent any number of input mechanisms, such as a microphone for speech, a touch-sensitive screen for gesture or graphical input, keyboard, mouse, motion input, speech and so forth. An output device can also be one or more of a number of output mechanisms. In some instances, multimodal systems can enable a user to provide multiple types of input to communicate with the computing system architecture. A communications interface can generally govern and manage the user input and system output. There is no restriction on operating on any particular hardware arrangement and therefore the basic features here may easily be substituted for improved hardware or firmware arrangements as they are developed.

The storage device is typically a non-volatile memory and can be a hard disk or other types of computer-readable media which can store data that are accessible by a computer, such as magnetic cassettes, flash memory cards, solid state memory devices, digital versatile disks, cartridges, random access memories (RAMs), read only memory (ROM), and hybrids thereof.

The storage device can include software modules for controlling the processor. Other hardware or software modules are contemplated. The storage device can be connected to the system bus. In one aspect, a hardware module that performs a particular function can include the software component stored in a computer-readable medium in connection with the necessary hardware components, such as the processor, bus, output device, and so forth, to carry out various functions of the disclosed technology.

Embodiments within the scope of the present disclosure may also include tangible and/or non-transitory computer-readable storage media or devices for carrying or having computer-executable instructions or data structures stored thereon. Such tangible computer-readable storage devices can be any available device that can be accessed by a general purpose or special purpose computer, including the functional design of any special purpose processor as described above. By way of example, and not limitation, such tangible computer-readable devices can include RAM, ROM, EEPROM, CD-ROM or other optical disk storage, magnetic disk storage or other magnetic storage devices, or any other device which can be used to carry or store desired program code in the form of computer-executable instructions, data structures, or processor chip design. When information or instructions are provided via a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable storage devices.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform tasks or implement abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

Other embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

In certain aspects, provided are one or more computer-readable media having stored thereon instructions for performing any of the steps of the methods of the present disclosure using any of the synthetic strands of the present disclosure. According to some embodiments, provided are the one or more computer-readable media of any of the systems of the present disclosure. For example, provided are one or more computer-readable media comprising instructions stored thereon, which when executed by one or more processors, cause the one or more processors to use one or more position indicator readers to determine the sequence of a nucleic acid by reading position indicators of a synthetic strand of the present disclosure during or subsequent to hybridization of the synthetic strand to the nucleic acid.

### Kits

Aspects of the present disclosure further include kits. In certain embodiments, the kits find use, e.g., in performing any of the methods of the present disclosure. According to some embodiments, a kit of the present disclosure includes a plurality of any of the second nucleic acids of the disclosure. In some embodiments, kits include surfaces having the second nucleic acids arranged thereon (e.g., in any of the configurations described herein, such as a chip).

A kit of the present disclosure may include one or more reagents that find use in sequencing nucleic acids using the synthetic strands. For example, a kit of the present disclosure may include a solution (e.g., a hybridization buffer solution) having a pH, salt concentration, one or more components (e.g., chelating agents), and/or the like useful for providing suitable conditions for contacting a nucleic acid to be sequenced with a second nucleic acid.

A kit of the present disclosure may further include instructions for performing any of the methods of the present disclosure, e.g., instructions for using electrolyzed nucleic acids to sequence a first nucleic acid. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### EMBODIMENTS

Notwithstanding the appended claims, the present disclosure is also defined by the following embodiments.

1. A method comprising:
causing relative movement of a first nucleic acid through an opening formed at least in part by an electrolyzed second nucleic acid; and
during the relative movement, detecting a varying conductance along the first nucleic acid indicative of sequential interactions between nucleobases of the first nucleic acid and one or more nucleobases of the electrolyzed second nucleic acid.

2. A method comprising:
causing relative movement of a first nucleic acid through an opening formed at least in part by an electrolyzed second nucleic acid; and
during the relative movement, detecting a varying conductance between the first nucleic acid and an electrode proximate to the first nucleic acid, wherein the varying conductance is indicative of sequential interactions between nucleobases of the first nucleic acid and one or more nucleobases of the electrolyzed second nucleic acid.

3. The method according to embodiment 1 or embodiment 2, wherein causing the relative movement comprises pulling the first nucleic acid through the opening formed at least in part by the electrolyzed second nucleic acid.

4. The method according to embodiment 3, wherein the first nucleic acid is attached to an elongate structure, and wherein pulling the first nucleic acid through the opening comprises pulling the elongate structure in the direction that the first nucleic acid is to be pulled.

5. The method according to embodiment 4, wherein the elongate structure comprises a nanotube, a nanowire, or a biopolymer.

6. The method according to embodiment 5, wherein the biopolymer is a nucleic acid.

7. The method according to embodiment 6, wherein the first nucleic acid and the nucleic acid comprise ends complementary to each other and are hybridized to each other during the pulling.

8. The method according to embodiment 1 or embodiment 2, wherein the electrolyzed second nucleic acid is disposed within a channel, and wherein causing the relative movement comprises translocating the first nucleic acid through the opening formed at least in part by the electrolyzed second nucleic acid within the channel.

9. The method according to any one of embodiments 1 to 8, wherein the electrolyzed second nucleic acid comprises first and second discontinuous regions attached to a surface such that the electrolyzed second nucleic acid forms a bridge structure, and wherein the relative movement of the first nucleic acid is through the opening of the bridge structure.

10. The method according to embodiment 9, wherein the surface is functionalized with oligonucleotides comprising sequences complementary to the first and second discontinuous regions, and wherein the first and second discontinuous regions are attached to the surface via hybridization to the oligonucleotides.

11. The method according to any one of embodiments 1 to 8, wherein the electrolyzed second nucleic acid comprises a first end attached to a surface and forms a stem-loop structure, and wherein the relative movement of the first nucleic acid is through the opening of the loop portion of the stem-loop structure.

12. The method according to any one of embodiments 1 to 8, wherein the electrolyzed second nucleic acid comprises a first end attached to a surface and first and second discontinuous regions hybridized to a third nucleic acid molecule such that the electrolyzed second nucleic acid and third nucleic acid molecule form a loop structure, and wherein the relative movement of the first nucleic acid is through the opening of the loop structure.

13. The method according to embodiment 11 or embodiment 12, wherein the first end of the electrolyzed second nucleic acid is attached to the surface via a biotin-streptavidin interaction or via magnetic attraction.

14. The method according to any one of embodiments 9 to 13, wherein causing the relative movement comprises moving the surface relative to the first nucleic acid.

15. The method according to embodiment 14, wherein the first nucleic acid is immobilized during the relative movement.

16. The method according to any one of embodiments 1 to 15, wherein the electrolyzed second nucleic acid is one of a plurality of electrolyzed nucleic acids, and wherein the method comprises causing relative movement of the first nucleic acid through a plurality of openings formed at least in part by the plurality of electrolyzed nucleic acids.

17. The method according to embodiment 16, wherein one or more of the plurality of openings comprise a single type of nucleobase independently selected from a nucleobase that base pairs with adenine, a nucleobase that base pairs with thymine or uracil, a nucleobase that base pairs with guanine, and a nucleobase that base pairs with cytosine.

18. The method according to embodiment 16 or embodiment 17, wherein one or more of the plurality of openings comprise abasic nucleotides.

19. The method according to any one of embodiments 1 to 18, wherein the varying conductance comprises conductance fingerprints for the different nucleobases in the first nucleic acid.

20. The method according to embodiment 19, further comprising determining the identity of one or more nucleotides of the first nucleic acid based on the varying conductance.

21. The method according to embodiment 19 or embodiment 20, further comprising determining a nucleotide sequence of the first nucleic acid based on the varying conductance.

22. The method according to any one of embodiments 1 to 21, wherein the first nucleic acid is selected from genomic DNA, complementary DNA (cDNA), or RNA.

23. A system, comprising:
one or more processors, and
one or more non-transitory computer-readable media comprising instructions stored thereon that cause the system to:
   monitor a varying conductance along a first nucleic acid, or a varying conductance between the first nucleic acid and an electrode proximate to the first nucleic acid, wherein the varying conductance is indicative of sequential interactions between nucleobases of a first nucleic acid and one or more nucleobases of an electrolyzed second nucleic acid.

24. The system of embodiment 23, wherein the varying conductance comprises conductance fingerprints for the different nucleobases in the first nucleic acid, and wherein the one or more non-transitory computer-readable media comprises instructions stored thereon that cause the system to determine the identity of one or more nucleotides of the first nucleic acid based on the varying conductance.

25. The system of embodiment 23 or embodiment 24, wherein the varying conductance comprises conductance fingerprints for the different nucleobases in the first nucleic acid, and wherein the one or more non-transitory computer-readable media comprises instructions stored thereon that cause the system to determine a nucleotide sequence of the first nucleic acid based on the varying conductance.

26. One or more non-transitory computer-readable media comprising instructions stored thereon that cause a system to:
monitor a varying conductance along a first nucleic acid, or a varying conductance between the first nucleic acid and an electrode proximate to the first nucleic acid, wherein the varying conductance is indicative of sequential interactions between nucleobases of a first nucleic acid and one or more nucleobases of an electrolyzed second nucleic acid.

27. The one or more non-transitory computer-readable media of embodiment 26, wherein the varying conductance comprises conductance fingerprints for the different nucleobases in the first nucleic acid, and wherein the one or more non-transitory computer-readable media comprises instructions stored thereon that cause the system to determine the identity of one or more nucleotides of the first nucleic acid based on the varying conductance.

28. The one or more non-transitory computer-readable media of embodiment 26 or embodiment 27, wherein the varying conductance comprises conductance fingerprints for the different nucleobases in the first nucleic acid, and wherein the one or more non-transitory computer-readable media comprises instructions stored thereon that cause the system to determine a nucleotide sequence of the first nucleic acid based on the varying conductance.

The following is offered by way of example and not by way of limitation:

### EXPERIMENTAL

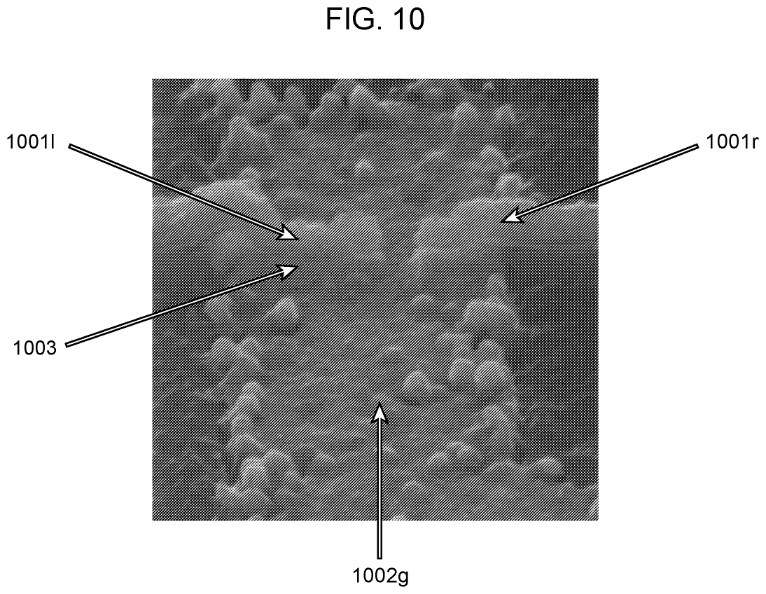

A chip for monitoring varying conductance associated with conductance fingerprints was constructed as shown in FIG. 9A-9B. A right and left electrode as well as a corresponding gate electrode (i.e., according to the structure shown in FIG. 9B) was imaged using a scanning helium ion microscope. The resulting micrograph is presented in FIG. 10. As shown in FIG. 10, left electrode 10011 and right electrode 1001r are insulated from gate electrode 1002g via insulating layer 1003.

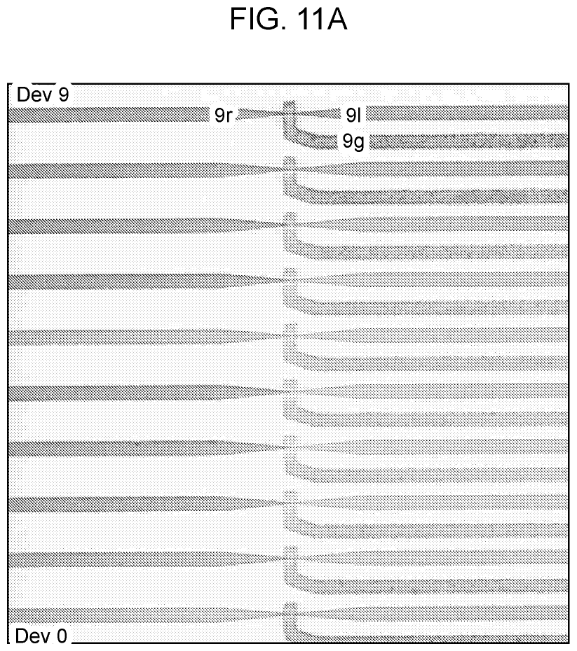

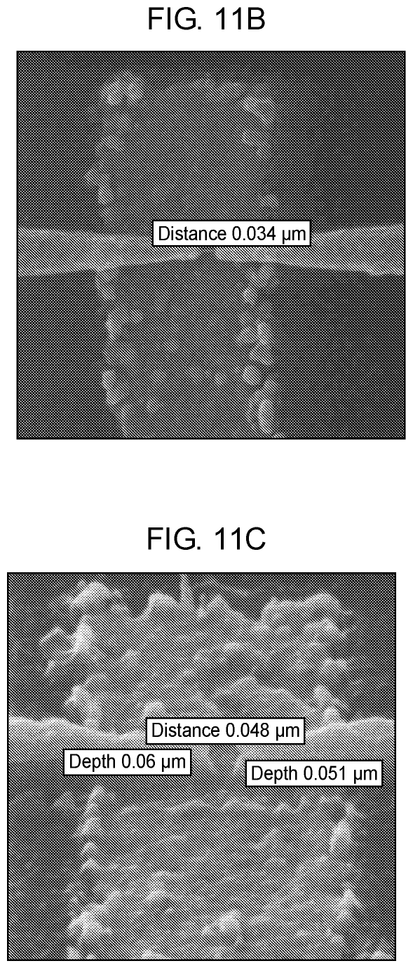

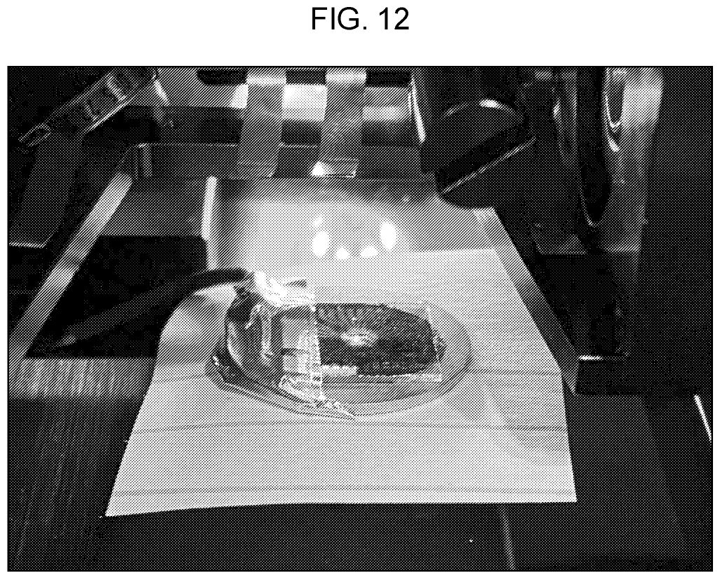

FIG. 11A-11C depict additional helium ion micrographs taken of the chip constructed as shown in FIG. 9A-9B. FIG. 11A shows a plurality of left and right electrodes and their respective gate electrodes arranged as discussed in FIG. 0.9B. FIG. 11B and FIG. 11C depict exemplary distances between left and right electrodes. In FIG. 11B, the distance is 34 nm. In FIG. 11C, the distance is 48 nm. FIG. 12 presents a prototype of a chip constructed as shown in FIG. 9A-9B.

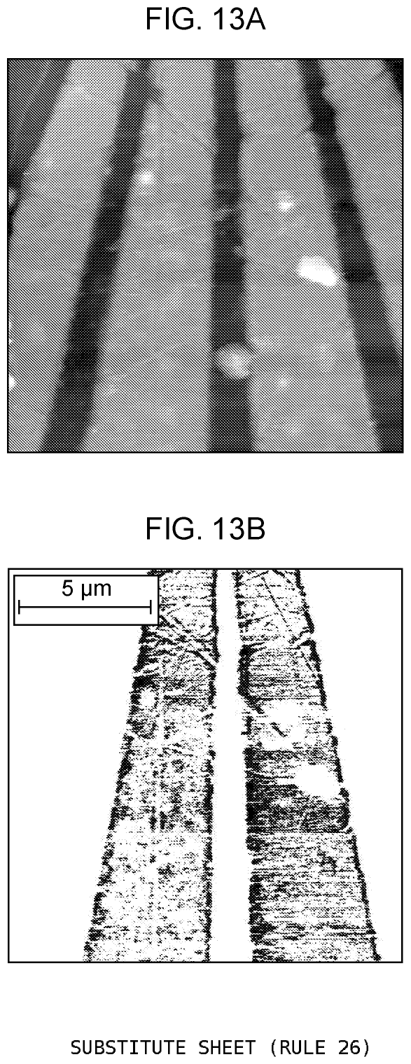

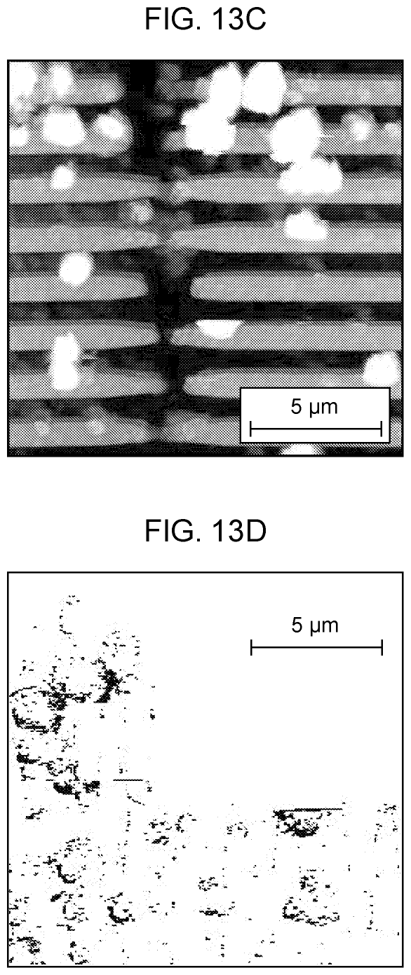

FIG. 13A-13D present images of the chip electrodes that were obtained via atomic force microscopy (AFM). The images in FIG. 13A and FIG. 13C were produced via AFM tapping mode, while the images of FIG. 13B and FIG. 13D were produced via conductive AFM and show that that the deposited electrodes can conduct current.

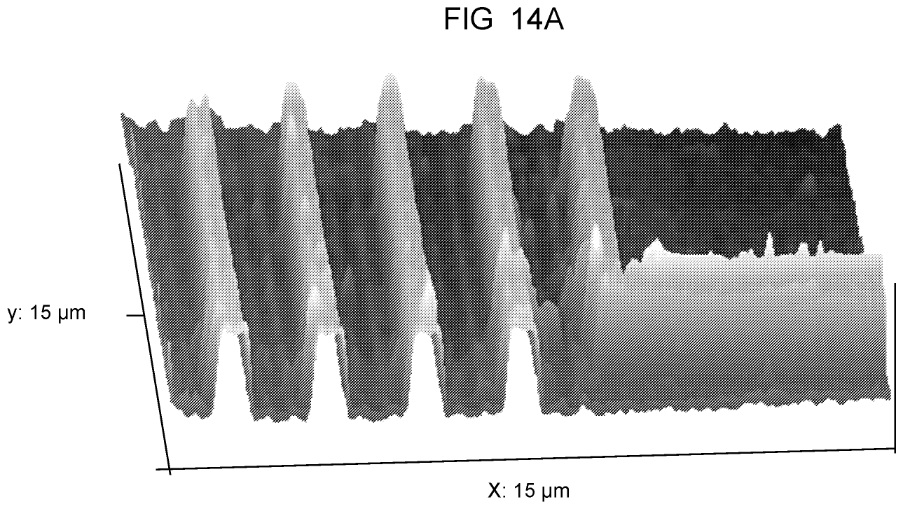

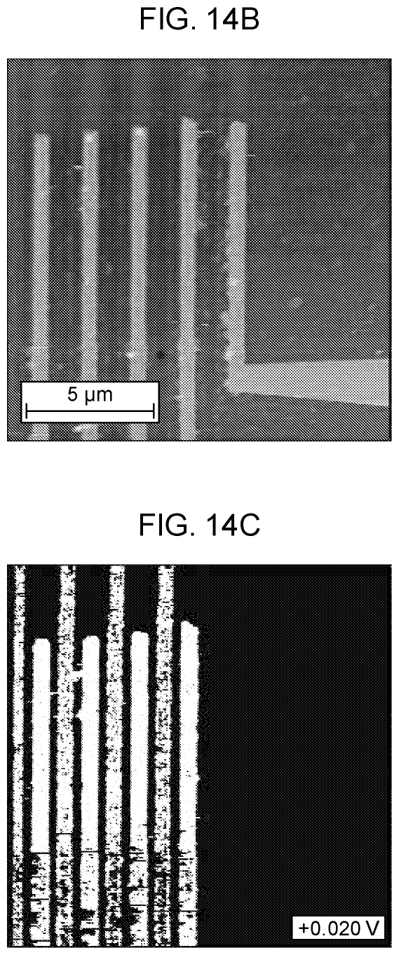

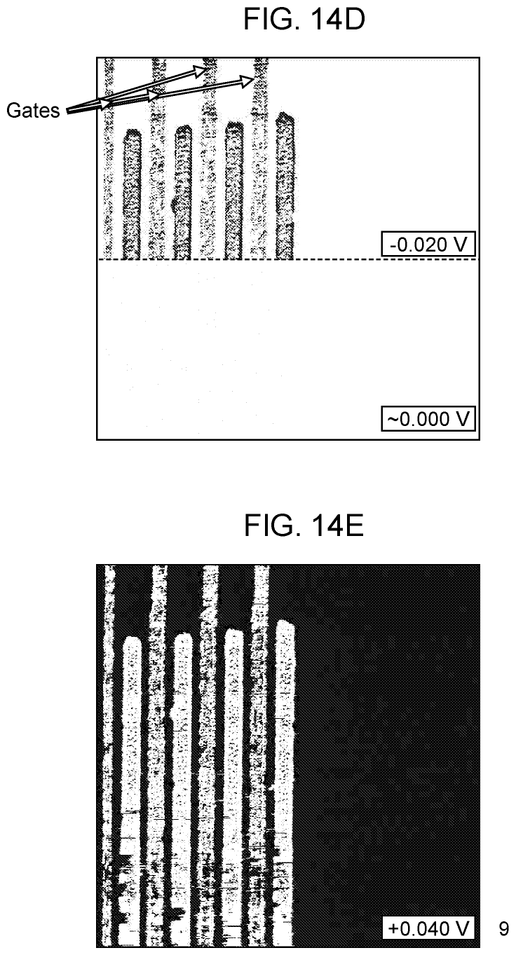

FIG. 14A-14E depict topographic and conductive aspects of the chip constructed as shown in FIG. 9A-9B. FIG. 14A shows a 3D topographic AFM image of the electrodes, while FIG. 14B presents a 2D image of the same. FIG. 14C-E show testing of the conductivity of the gold electrodes using conductive AFM. While the topographic tapping mode images readily show the electrodes, they miss the gates as these structure are below the surface. Nonetheless, conductive AFM is able to resolve the gate as conductive (white=current=conductive).

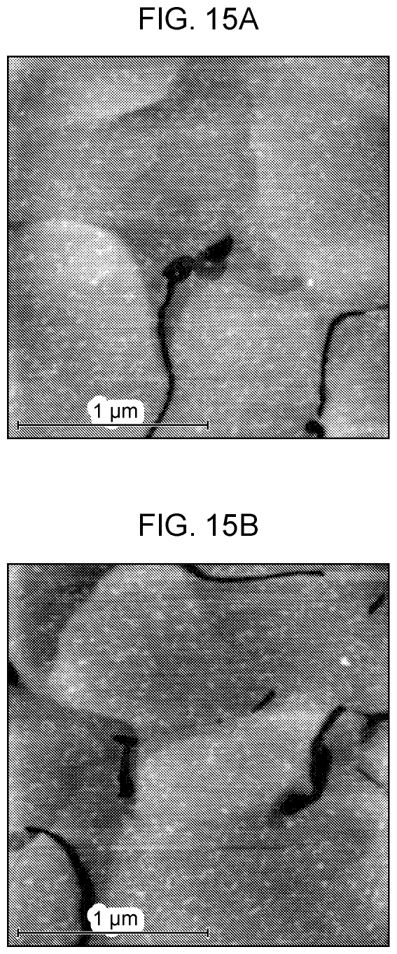

FIG. 15A-15B present AFM images showing single stranded DNA immobilized on a gold substrate via thio bonds. The “worm-like” structures are the DNA. These images demonstrate that DNA may be immobilized on gold electrodes in a manner permitting the formation of electrolyzed bridge structures that find use in practicing methods according to embodiments of the present disclosure.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method comprising:
causing relative movement of a first nucleic acid through an opening formed at least in part by an electrolyzed second nucleic acid; and
during the relative movement, detecting a varying conductance along the first nucleic acid indicative of sequential interactions between nucleobases of the first nucleic acid and one or more nucleobases of the electrolyzed second nucleic acid.

2. A method comprising:
causing relative movement of a first nucleic acid through an opening formed at least in part by an electrolyzed second nucleic acid; and
during the relative movement, detecting a varying conductance between the first nucleic acid and an electrode proximate to the first nucleic acid, wherein the varying conductance is indicative of sequential interactions between nucleobases of the first nucleic acid and one or more nucleobases of the electrolyzed second nucleic acid.

3. The method according to claim 1 or claim 2, wherein causing the relative movement comprises pulling the first nucleic acid through the opening formed at least in part by the electrolyzed second nucleic acid.

4. The method according to claim 3, wherein the first nucleic acid is attached to an elongate structure, and wherein pulling the first nucleic acid through the opening comprises pulling the elongate structure in the direction that the first nucleic acid is to be pulled.

5. The method according to claim 4, wherein the elongate structure comprises a nanotube, a nanowire, or a biopolymer.

6. The method according to claim 5, wherein the biopolymer is a nucleic acid.

7. The method according to claim 6, wherein the first nucleic acid and the nucleic acid comprise ends complementary to each other and are hybridized to each other during the pulling.

8. The method according to claim 1 or claim 2, wherein the electrolyzed second nucleic acid is disposed within a channel, and wherein causing the relative movement comprises translocating the first nucleic acid through the opening formed at least in part by the electrolyzed second nucleic acid within the channel.

9. The method according to any one of claims 1 to 8, wherein the electrolyzed second nucleic acid comprises first and second discontinuous regions attached to a surface such that the electrolyzed second nucleic acid forms a bridge structure, and wherein the relative movement of the first nucleic acid is through the opening of the bridge structure.

10. The method according to claim 9, wherein the surface is functionalized with oligonucleotides comprising sequences complementary to the first and second discontinuous regions, and wherein the first and second discontinuous regions are attached to the surface via hybridization to the oligonucleotides.

11. The method according to any one of claims 1 to 8, wherein the electrolyzed second nucleic acid comprises a first end attached to a surface and forms a stem-loop structure, and wherein the relative movement of the first nucleic acid is through the opening of the loop portion of the stem-loop structure.

12. The method according to any one of claims 1 to 8, wherein the electrolyzed second nucleic acid comprises a first end attached to a surface and first and second discontinuous regions hybridized to a third nucleic acid molecule such that the electrolyzed second nucleic acid and third nucleic acid molecule form a loop structure, and wherein the relative movement of the first nucleic acid is through the opening of the loop structure.

13. The method according to claim 11 or claim 12, wherein the first end of the electrolyzed second nucleic acid is attached to the surface via a biotin-streptavidin interaction or via magnetic attraction.

14. The method according to any one of claims 9 to 13, wherein causing the relative movement comprises moving the surface relative to the first nucleic acid.

15. The method according to claim 14, wherein the first nucleic acid is immobilized during the relative movement.

16. The method according to any one of claims 1 to 15, wherein the electrolyzed second nucleic acid is one of a plurality of electrolyzed nucleic acids, and wherein the method comprises causing relative movement of the first nucleic acid through a plurality of openings formed at least in part by the plurality of electrolyzed nucleic acids.

17. The method according to claim 16, wherein one or more of the plurality of openings comprise a single type of nucleobase independently selected from a nucleobase that base pairs with adenine, a nucleobase that base pairs with thymine or uracil, a nucleobase that base pairs with guanine, and a nucleobase that base pairs with cytosine.

18. The method according to claim 16 or claim 17, wherein one or more of the plurality of openings comprise abasic nucleotides.

19. The method according to any one of claims 1 to 18, wherein the varying conductance comprises conductance fingerprints for the different nucleobases in the first nucleic acid.

20. The method according to claim 19, further comprising determining the identity of one or more nucleotides of the first nucleic acid based on the varying conductance.

21. The method according to claim 19 or claim 20, further comprising determining a nucleotide sequence of the first nucleic acid based on the varying conductance.

22. The method according to any one of claims 1 to 21, wherein the first nucleic acid is selected from genomic DNA, complementary DNA (cDNA), or RNA.

23. A system, comprising:
one or more processors; and
one or more non-transitory computer-readable media comprising instructions stored thereon that cause the system to:
monitor a varying conductance along a first nucleic acid, or a varying conductance between the first nucleic acid and an electrode proximate to the first nucleic acid, wherein the varying conductance is indicative of sequential interactions between nucleobases of a first nucleic acid and one or more nucleobases of an electrolyzed second nucleic acid.

24. The system of claim 23, wherein the varying conductance comprises conductance fingerprints for the different nucleobases in the first nucleic acid, and wherein the one or more non-transitory computer-readable media comprises instructions stored thereon that cause the system to determine the identity of one or more nucleotides of the first nucleic acid based on the varying conductance.

25. The system of claim 23 or claim 24, wherein the varying conductance comprises conductance fingerprints for the different nucleobases in the first nucleic acid, and wherein the one or more non-transitory computer-readable media comprises instructions stored thereon that cause the system to determine a nucleotide sequence of the first nucleic acid based on the varying conductance.

26. One or more non-transitory computer-readable media comprising instructions stored thereon that cause a system to:
monitor a varying conductance along a first nucleic acid, or a varying conductance between the first nucleic acid and an electrode proximate to the first nucleic acid, wherein the varying conductance is indicative of sequential interactions between nucleobases of a first nucleic acid and one or more nucleobases of an electrolyzed second nucleic acid.

27. The one or more non-transitory computer-readable media of claim 26, wherein the varying conductance comprises conductance fingerprints for the different nucleobases in the first nucleic acid, and wherein the one or more non-transitory computer-readable media comprises instructions stored thereon that cause the system to determine the identity of one or more nucleotides of the first nucleic acid based on the varying conductance.

28. The one or more non-transitory computer-readable media of claim 26 or claim 27, wherein the varying conductance comprises conductance fingerprints for the different nucleobases in the first nucleic acid, and wherein the one or more non-transitory computer-readable media comprises instructions stored thereon that cause the system to determine a nucleotide sequence of the first nucleic acid based on the varying conductance.
